(19) United States
(12) Patent Application Publication
(10) Pub. No.: US 2019/0350741 A1
(43) Pub. Date: Nov. 21, 2019

(54) ABDOMINAL SUPPORT TRUSS

(71) Applicant: Douglas Graf, Maple Heights, OH (US)

(72) Inventor: Douglas Graf, Maple Heights, OH (US)

(21) Appl. No.: 16/418,141

(22) Filed: May 21, 2019

Related U.S. Application Data
(60) Provisional application No. 62/674,098, filed on May 21, 2018.

(51) Int. Cl. A61F 5/28

(52) U.S. Cl. CPC A61F 5/28; A61F 5/30

(57) ABSTRACT

An abdominal support truss. The abdominal support truss includes a first belt and a second belt. The first belt is configured to encircle a belly of the user. The second belt is configured to encircle a waist of the user. A pad is attached to an interior surface of the second belt. The pad is configured to compress and to rest against a surgical site. A ratcheting system is operably disposed on the second belt. Engagement of the ratcheting system tightens the second belt.

### CROSS REFERENCE TO RELATED APPLICATIONS

This application claims the benefit of U.S. Provisional Application No. 62/674,098 filed on May 21, 2018. The above identified patent application is herein incorporated by reference in its entirety to provide continuity of disclosure.

### BACKGROUND OF THE INVENTION

The present invention relates to an abdominal support truss. More specifically, the present invention provides an apparatus designed to increase the comfort of patients recovering from surgical operations where a site can be made more comfortable by using a pad.

A hernia occurs when an organ or fatty tissue squeezes through a weak spot in a muscle or connective tissue. The most common type of hernia is an inguinal hernia, however other types of hernias also exist. An inguinal hernia exists where the intestines extend through a weak spot in the lower abdominal wall, commonly in the inguinal canal. Hernias often require surgery. During these surgeries, incisions are made in the area of the hernia. The hernia is then placed back into the position it is supposed to be in, tied off, or is removed. The weak spot in the muscle or connective tissue is then sutured or otherwise reinforced, so as to prevent the hernia from returning. During the post-surgery period following a hernia procedure, structural support may be helpful to promote healing and comfort to the patient.

Other surgeries require similar post-surgery support as well. For example, when a Caesarian section is performed, a wound is created that can benefit from structural support. Appendectomies also involve the surgical opening of the abdominal cavity in order to remove the appendix. Even observatory procedures, such as laparoscopies, create an incision that may be sensitive to movements from the patient. Thus, there is a defined need amongst the known art for a surgical device that provides post-surgical support to patients who have undergone abdominal procedures and may be vulnerable to sutures reopening, or other post-operative problems.

### SUMMARY OF THE INVENTION

In view of the foregoing disadvantages inherent in the known types of hernia support apparatuses now present in the prior art, the present invention provides an abdominal support truss wherein the same can be utilized for providing convenience for the user when providing post-operative support to a patient of an abdominal surgical procedure.

The present system comprises a first belt and a second belt. The first belt is configured to encircle a belly of the user. The second belt is configured to encircle a waist of the user. A pad is attached to an interior surface of the second belt. The pad is configured to compress and to rest against a surgical site. A ratcheting system is operably disposed on the second belt. Engagement of the ratcheting system tightens the second belt.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the abdominal support truss.

FIG. 2 shows a perspective view of a pad of an embodiment of the abdominal support truss.

FIG. 3 shows a perspective view of a second belt and a ratcheting system of an embodiment of the abdominal support truss.

FIG. 4 shows a perspective view of a first belt of an embodiment of the abdominal support truss.

### DETAILED DESCRIPTION OF THE INVENTION

Reference is made herein to the attached drawings. Like reference numerals are used throughout the drawings to depict like or similar elements of the abdominal support truss. The figures are intended for representative purposes only and should not be considered to be limiting in any respect.

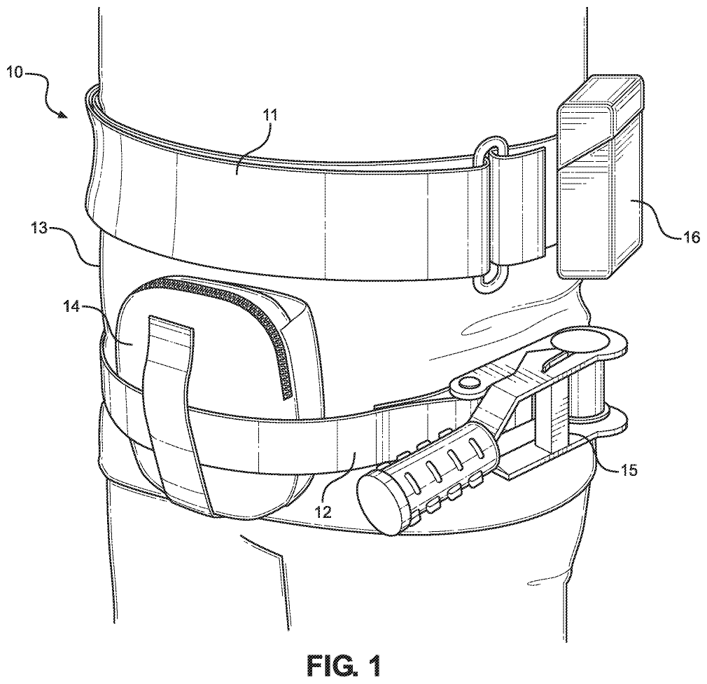

Referring now to FIG. 1, there is shown a perspective view of an embodiment of the abdominal support truss. The abdominal support truss 10 comprises a first belt 11 and a second belt 12. The first belt 11 is configured to encircle a belly of a user 13. The first belt 11 is flexible and is made of any suitable material. In one embodiment, the first belt 11 is made of a nylon material. The first belt 11 is adjustable around the belly of the user 13, such that support is provided to the belly of the user 13, while also providing a comfortable fit around the belly of the user 13. In an alternate embodiment, the first belt 11 comprises a padded material on an internal surface of the first belt, such that enhanced comfort is provided to the user 13.

The second belt 12 is flexible and is made of any suitable material. The second belt 12 is configured to encircle a waist of the user 13. A pad 14 is affixed to an interior surface of the second belt 12. In the illustrated embodiment, the pad 14 is slidably disposed upon the second belt 12, such that the position of the pad 14 is adjustable by the user 13. Specifically, the pad 14 is configured to press against a surgical site, such that comfort is provided to the surgical site via the pad 14. In one embodiment, the pad 14 is made of a foam material. In alternate embodiments, the pad 14 is made of any material suitable for providing comfort to a surgical site, such as a cushioned fabric or a gel pad, for example.

A ratcheting system 15 is operably disposed on the second belt 12. Engagement of the ratcheting system 15 tightens the second belt 12. The second belt 12 includes a first end and a second end secured to the ratcheting system 15 such that the first end is permanently and unmovably affixed to the ratcheting system 15 and the second end is operably fed through the ratcheting system 15. As such, the user 13 sets the circumference of the second belt 12 by feeding the second end of the second belt 12 through the ratcheting system 15, then locking the circumference of the second belt 12 by placing the ratcheting system 15 in a closed position.

In the illustrated embodiment, the first belt 11 further comprises a pouch 16 thereon. The pouch 16 is configured to hold an object provided by the user. For example, the pouch 16 may be dimensioned to hold a medication container. As such, the individual wearing the abdominal support truss 10 will be able to access his or her medication without having to engage in excessive movement. The pouch 16 may be movable around the first belt 11 or the second belt 12, such that the user can more easily access the pouch 16 depending on the location or nature of his or her surgical site.

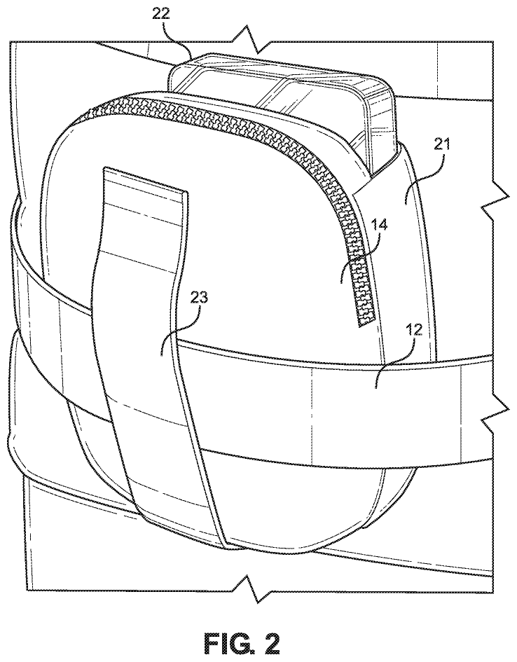

Referring now to FIG. 2, there is shown a perspective view of a pad of an embodiment of the abdominal support truss. In the illustrated embodiment, the pad 14 comprises a housing having a sealable opening. As such, a padded material can be disposed in the sealable opening such that the level of support provided by the pad 14 is adjustable by the user. For example, a user may desire to use a firmer pad or a softer pad depending upon the nature of their surgery or recovery.

In the illustrated embodiment, the pad 14 comprises a pocket 21. The pocket 21 is dimensioned to receive an object, such as an ice pack 22. The use of an ice pack 22 allows for enhanced comfort to be provided to the user as the surgical area is cooled by the ice pack 22. Other objects, such as a warming pad or an additional comfort pad may be inserted into the pocket to provide for enhanced comfort and usability. Different pads of differing densities may be utilized for a desired amount of support or comfort.

In the illustrated embodiment, the pad 14 is secured to the second belt 12 via a strap 23. By securing the pad 14 to the second belt 12 via the strap 23, the pad 14 is slidable along the second belt 12. As such, the position of the pad 14 can be laterally adjusted by the user to rest upon a surgical site as desired by the user. For example, a user who has undergone a hernia procedure will need the pad 14 to rest in a different location than a user who has undergone a hip procedure.

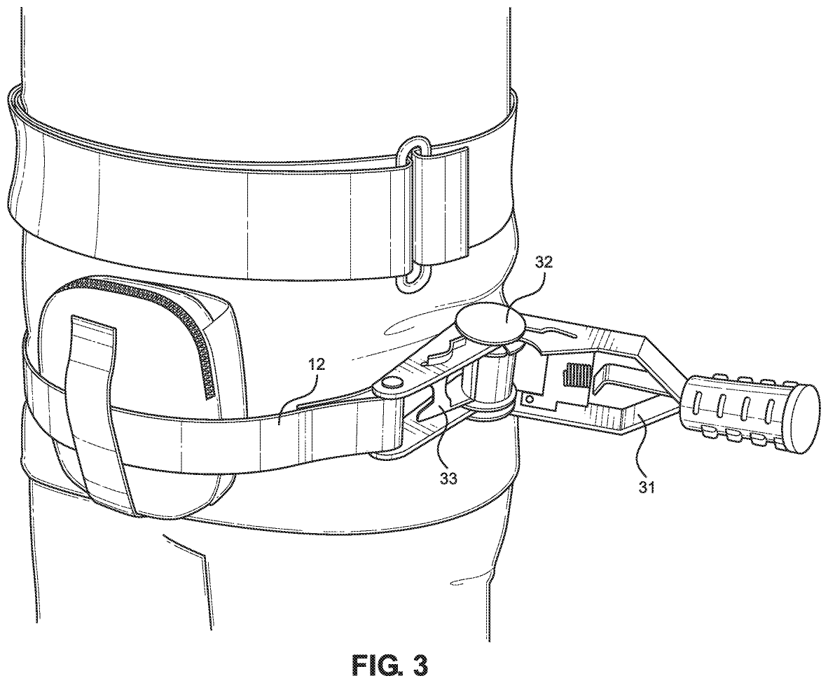

Referring now to FIG. 3, there is shown a perspective view of a second belt and a ratcheting system of an embodiment of the abdominal support truss. In the illustrated embodiment, the ratcheting system comprises a handle 31 in operable connection with a ratchet 32. The ratchet 32 and the handle 31 are disposed upon a frame 33. The frame 33 is affixed on a first end to the second belt 12 and is removably affixed at a second end to the second belt 12. As such, the second belt 12 is adjustable around the waist of the user.

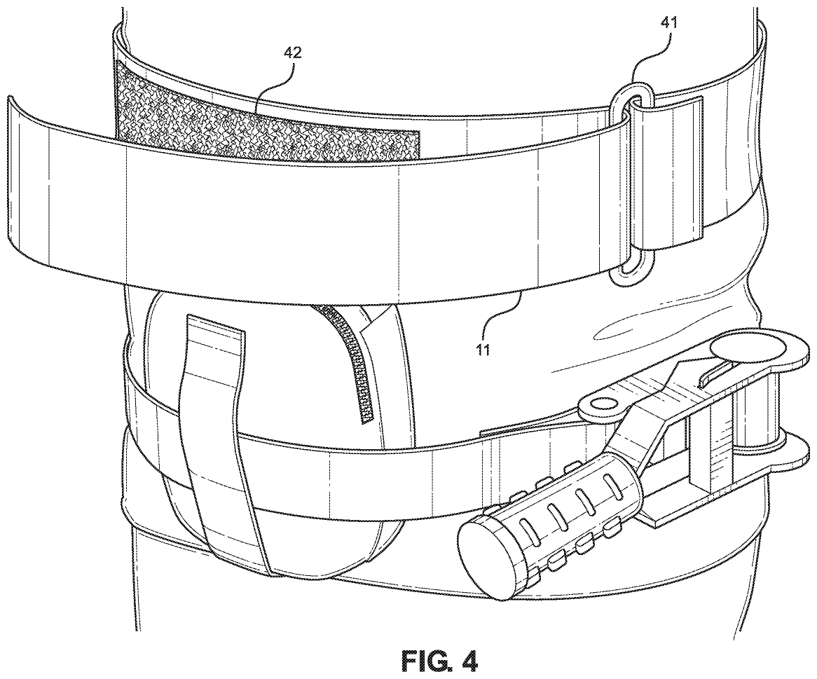

Referring now to FIG. 4, there is shown a perspective view of a first belt of an embodiment of the abdominal support truss. In the illustrated embodiment, the first belt 11 comprises a loop 41 thereon. The loop 41 is configured to allow for adjustment of the first belt 11. As shown, the loop 41 defines a slot, wherein the slot is dimensioned to receive the first belt 11. The first belt 11 is permanently secured at one end to the loop 41 and is slidably engaged with the loop 41 at the other end.

In the shown embodiment, the first belt 11 further comprises a hook and loop fastener 42 thereon. The hook and loop fastener 42 is configured to hold the first belt 11 in a wrapped position around the waist of the user. Specifically, in the illustrated embodiment, a loop portion of the hook and loop fastener 42 and a hook portion of the hook and loop fastener 42 are disposed on an external surface of the first belt 11. As such, the loop portion and the hook portion are engaged when pressed together after the first belt 11 passes through the loop 41 on the first belt 11. Thus, the first belt 11 can be secured as tightly or as loosely as desired by the user.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An abdominal support truss, comprising:
a first belt and a second belt;
the first belt configured to encircle a belly of a user;
the second belt configured to encircle a waist of the user;
a pad affixed to an interior surface of the second belt;
the pad configured to compress and rest against a surgical site;
a ratcheting system operably disposed on the second belt;
wherein engagement of the ratcheting system tightens the second belt.

2) The abdominal support truss of claim 1, wherein the first belt further comprises a loop disposed thereon, wherein the loop is configured to allow for adjustment of the first belt.

3) The abdominal support truss of claim 1, wherein a hook and loop fastener is disposed on the first belt.

4) The abdominal support truss of claim 1, wherein a pocket is disposed on an interior surface of the pad.

5) The abdominal support truss of claim 1, wherein the pad is made of a foam material.

6) The abdominal support truss of claim 1, wherein the ratcheting system comprises a handle and a ratchet, wherein the ratcheting system is movable between an open position and a closed position.
